(19) United States
(12) Patent Application Publication
(10) Pub. No.: US 2020/0046253 A1
(43) Pub. Date: Feb. 13, 2020

(54) INTERCEPT SYSTEM FOR CONTROLLED SUBSTANCES

(71) Applicant: CANNABIX TECHNOLOGIES INC., Burnaby (CA)

(72) Inventors: Rajpaul ATTARIWALA, Vancouver (CA); Kulwant MALHI, Delta (CA)

(21) Appl. No.: 16/344,578

(22) PCT Filed: Oct. 30, 2017
(86) PCT No.: PCT/CA2017/000233

§ 371 Date: Apr. 24, 2019

Related U.S. Application Data
(60) Provisional application No. 62/524,938, filed on Jun. 26, 2017.
(60) Provisional application No. 62/414,286, filed on Oct. 28, 2016.

(51) Int. Cl. A61B 5/08; A61B 5/091; A61B 5/097; B60K 28/06

(52) U.S. Cl. CPC A61B 5/082; A61B 5/091; G08B 21/182; B60K 28/063; A61B 5/097

(57) ABSTRACT

A controlled substance Intercept system for collecting and detecting levels of one or more controlled substances in the breath of a subject and intercepting electric operation of a device, comprising: a sample capture module; a THC identification module; and an intercept module. The THC identification module sends an electric or optical signal to the intercept module if a level of controlled substance present in a sample captured in the sample capture, module exceeds a set threshold, for use in applications to enable or disable operation of equipment such as motor vehicles and construction equipment, and to enable or disable access to locations where impairment would pose a hazard, such as air or marine traffic control centers.

### TECHNICAL FIELD OF THE: INVENTION

The present invention relates to detection devices for cannabis and other controlled substances.

The present invention is directed to an ignition interlock or other intercept device for the detection of cannabis and other controlled substances from the exhaled breath of a subject and prevention of operation of equipment or access to locations.

### BACKGROUND OF THE INVENTION

Impaired driving is the cause of many injuries and deaths. Many jurisdictions have imposed requirements for ignition interlock devices for motor vehicles that are operated by those who have been arrested or convicted of driving under the influence of alcohol. These systems typically require the vehicle operator to take a breath test by blowing into a mouthpiece installed in the car. The mouth piece measures the amount of alcohol in the vehicle operator's breath and uses that measurement to determine the vehicle operator's blood alcohol content. This breath method has proven reliable and is accepted by legal systems as a noninvasive method to quantify alcohol levels.

Whereas alcohol is a simple molecule which can be examined directly by an exhaled breath exam, most commonly by exhaling into an ion spectroscopy chamber, detection of cannabis and other controlled substances is commonly performed by urine, blood, or oral specimen sampling. These methods are frequently invasive and require complicated devices for analysis.

The present invention seeks to expand the scope of vehicles ignition interlock devices to detect cannabis and other controlled substances using a breathalyzer device, and to use an intercept system to prevent operation of a vehicle or other machinery, and block access to locations where controlled substance levels must not exceed a set threshold.

In this specification, “controlled substances” means any legal or illicit consumable drug, chemical, or other substance which is controlled by government regulation, and includes cannabis and alcohol. The terms “controlled substance(s)” and “drug(s)” are used interchangeably throughout.

Detection of drugs by an exhaled breath method has been proposed; however, the prior art technique proposed is generic and non-selective for multiple different illicit drugs. The proposed devices have been described for detection of drugs within the exhaled breath using a fluid collection or filtration system. Most of these devices describe a tube into which the subject exhales, which indiscriminately collects molecules of interest in either a liquid or filtration device. This liquid or fluid trapped within a filter is then sent to a laboratory for analysis, which may take several days. These devices lack methods for measurement of exhaled breath volumetric flowrate and for regulating pressure. With exhaled breath, each individual has the capability to exhale to different pressures, and if, for example, a filter based system is utilized to measure cannabis, an unregulated high pressure provided by a subject can tear apart the filter. Also, as each subject has a different amount of exhaled breath, it is important to be able to quantity the flowrate, or total volumetric flow, that has been breathed into the device.

The prior art devices which are designed to measure exhaled breath describe a mouthpiece which comprises a tube into which the subject exhales. This tube without a rebreather valve, requires that the subject inhale through their nose, or remove the device from their lips to inhale. Prior devices designed for exhaled breath do not describe a method of removal of fluid or solid contaminants, such as by way of a spit trap to collect or remove oral fluids, which prevents oral fluid contaminants from reaching a gas material detection chamber.

### BRIEF DESCRIPTION OF THE DRAWINGS

A detailed description of the preferred embodiments is provided below by way of example only and with reference to the following drawings, in which:

FIG. 1 is a schematic illustration showing the ignition interlock system architecture of the present invention;

FIG. 2 is a schematic illustration showing the ignition interlock system architecture of the present invention for use in a vehicle immobilizer; and

FIG. 3 is a schematic illustration showing the ignition interlock architecture of the present invention with a voice print recognition feature.

In the drawings, one embodiment of the invention is illustrated by way of example. It is to be expressly understood that the description and drawings are only for illustration and as an aid to understanding, and are not intended as a definition of the limits of the invention.

### SUMMARY OF THE INVENTION

The present invention provides drug detection technologies as applied to enable or disable operation of equipment, and to enable or disable, access to locations where impairment would pose a hazard. As examples, this disclosure describes driving a car, operating a piece of construction equipment, or acting as controller for air or marine traffic, but other equipment control is within the scope of the invention.

The present invention comprises a system which is designed to measure a subject's breath, remove fluid and/or contaminants, and collect a sample of tetrahydrocannabinol (THC) or other controlled substance in an entrapment container for detection of cannabis or other controlled substance use, and to prevent operation of a vehicle or other machinery, or access to a location, if levels exceed set limits.

The invention provides a controlled substance intercept system for detecting levels of one or more controlled substances in the breath of a subject and intercepting electric operation of a device, comprising: a sample capture module; a THC identification module; and an intercept module; wherein the THC identification module sends an electric or optical signal to the intercept module if a level of controlled substance present in a sample captured in the sample capture module exceeds a set threshold.

The controlled substance intercept system may include an indicator module, which may be lights, display assemblies, sirens, horns, speakers, and vibrating devices.

The sample capture module may comprise means for removal of water from the breath, means for measuring the volume of the breath, and valve means for preventing withdrawal of the breath sample by the subject during inhalation. The sample capture module may include a pressure regulator for regulating the pressure of the breath introduced into the system, which may be an inflatable balloon to regulate the pressure of the breath introduced into the system.

The THC identification module may comprise a controlled substance detection method such as Asymmetric Waveform for Mobility Spectrometry (FANS), Mass Spectrometry, Liquid Chromatography, Ion Resonance Chamber, Magnetic Resonance Chamber, Light Spectroscopy, Color detection, and Filtering.

The intercept module may comprise means for intercepting the flow of electricity in a device, and may be a vehicle immobilizer. The intercept module may include an electronic door lock. The intercept module is remote from the sample capture module and THC identification module.

The controlled substance intercept system may include a voice recognition component for subject identification confirmation.

In another aspect the invention provides a method of intercepting equipment operation in response to the presence of cannabis or other controlled substance by analyzing breath of a subject comprising the steps of: removing contaminant material from the breath; collecting a sample of at least one component of the breath of the subject after the contaminant material has been removed therefrom; analyzing the sample for the presence of one or more controlled substances; sending a control signal to an immobilizer if the controlled substance is detected; and immobilizing the equipment.

The method may include additional steps or allowing the subject to inhale and exhale while introducing breath into the system, regulating the pressure of the breath introduced into the system, or measuring a volumetric amount of breath introduced into the system by the subject.

The invention includes a breathing tube with a rebreather valve, and a fluid and solid contaminant removal device. A pressure or volumetric flow measurement and regulating device is placed along the path of the exhaled breath pathway before or after a fixed or removable drug sample collecting chamber. The drug collecting chamber may contain a port for detection, preparation or collection of a sample of cannabis or other controlled substance. The device is designed to permit detection of cannabis use relatively quickly, thus allowing it to be used, for example, by police officers in the field, etc.

There is provided a controlled substance detection system for detecting one or more controlled substances in the breath of a subject, comprising: a containment trap for removing interfering materials from the breath of the subject; and a collection component for sampling components of breath introduced into the system through the containment trap for analysis to determine the presence of one or more controlled substances in the breath, in which the components of the breath sampled by the collection component are analyzed. The detection system may include a non-rebreather valve that allows the subject to inhale and exhale while introducing breath into the system, a pressure regulator for regulating the pressure of the breath introduced into the system, a flow measurement device for measuring a volumetric amount of breath introduced into the system by the subject, a mouthpiece for facilitating introduction of breath into the system by the subject, and a sampling port for allowing access to the components sampled by the collection component. The collection component may include a filter for trapping components of the breath sampled by the collection component. The containment trap may be a flap valve covering holes to allow fluids to be removed from the breath introduced into the system. The pressure regulator may be a balloon that inflates to regulate the pressure of breath introduced into the system. There may also be an appearance change material that changes appearance when in contact with a controlled substance.

The invention also provides a method of detecting cannabis or other controlled substance used by analyzing breath of a subject, comprising the steps of: removing contaminant material from the breath; collecting a sample of at least one component of the breath of the subject after the contaminant material has been removed therefrom; and analyzing the sample for the presence of one or more controlled substances. The method may include one or more of the additional steps of allowing the subject to inhale and exhale while introducing breath into the system, regulating the pressure of the breath introduced into the system, or measuring a volumetric amount of breath introduced into the system by the subject.

The invention further provides a detection system for detecting one or more controlled substances in the breath of a subject, comprising: a mouthpiece for facilitating introduction of breath into the system by the subject; a non-rebreather valve that allows the subject to inhale and exhale while introducing breath into the system; a containment trap for removing interfering materials from the breath of the subject introduced into the system; a pressure regulator for regulating the pressure of the breath introduced into the system a flow measurement device for measuring a volumetric amount of breath introduced into the system by the subject; a collection component for sampling components of breath introduced into the system through the containment trap: and a sampling port for allowing access to the components sampled by the collection component to facilitate analysis of the components sampled by the collection component to determine the presence of one or more controlled substances in the breath.

### DETAILED DESCRIPTION OF THE INVENTION

There are provided several innovative methods to support detecting and quantifying the presence of THC in a volume of breath exhaled by an individual.

The prior art discloses devices which sense the presence of alcohol through analyzing breath samples, then determine if a vehicle driver is under undue influence of alcohol, and, if found to exceed a pre-set threshold, then to prevent the vehicle from being started, or giving audible and visual signals to alert the driver, passengers and the public, that the vehicle in motion poses a danger. While it is possible, without too much technical difficulty, to bypass the disabling function of equipment currently in use, effective means to deter from such behavior involve the continuous monitoring of electric and movement related signals, logging them in the immobilizer's memory, and making them available to enforcement agencies. If the vehicle was operated without a breath sample having been given the combination of monitored signals would tell and the violation of the driving restriction thereby be proven.

First, before any such detection or measurement of THC in breath can typically succeed, those constituents that are contained in breath, and which potentially interfere with the measurement, predominantly water from saliva, need to he removed. Second, to gain the total amount of THC per volume of breath, which is used as a measure of impairment, the volume of breath must be determined, independent of how fast or slow, deep or shallow the breathing is being delivered. Third, to avoid any dilution of the breath sample by intermittent breath, delivery, a valve mechanism in a breath sample collection device is necessary to allow the individual to breathe in and out without taking the sample.

These three requirements are covered in Patent Application US 2015/0305651 A1, assigned to Cannabix Breathalyzer Inc., and incorporated by reference herein. In this specification this technology building block is referred to as a “Sample Capture Module”.

Once a conditioned breath sample is available it needs to be evaluated as to its contents of THC. Methods of evaluation include Asymmetric Waveform Ion Mobility Spectrometry (FAIMS), Mass Spectrometry, Liquid Chromatography, Ion Resonance Chamber, Magnetic Resonance Chamber, Light Spectroscopy, Color detection, and Filtering, Once the amount of THC contained in each, conditioned breath sample has been determined it can be compared against a threshold value, THC amounts exceeding the threshold value can be interpreted as there being intolerable danger of impairment to perform a certain function by the person who delivered the breath sample. This building block is referred to herein as “THC Identification Module”.

The information whether there is or there isn't the danger of impairment can now he used further. In some cases, displaying the result on a screen is all that is required. A roadside breathalyser would be an example. This building block is referred to herein as “Indicator Module”.

Other applications require the information to be available as a signal which can be processed to either pass or block energizing the ignition and/or the starter of an internal combustion engine. The latter would be the case of the car immobilizer. A crane operator at a construction site could create significant harm if operating the equipment while being impaired. Here the control signals to the typically electrical motors would need to be intercepted by an appropriate immobilizer device which receives the go/no-go signal from the THC Identification Module. Yet another case can be access to a building or work place where impairment of the person entering would create intolerable danger to people or equipment. The current sent to a door lock from the device which controls access through the door needs to be intercepted. Now, for a person identified as being authorized to pass through the door the actual door opening may only happen when the THC Identification Module sends the signal that no impairment has been detected. This building block is referred to herein as “Intercept Module”.

As illustrated in FIG. 1, the Sample Capture Module delivers a sufficiently contaminant- free breath sample of known volume to the THC Identification Module which evaluates whether the amount of THC contained per breath volume is below, at, or above a pre-determined threshold, and delivers this result in the form of an appropriate electrical signal to further an indicator Module and/or Intercept Modules or Intercept Devices.

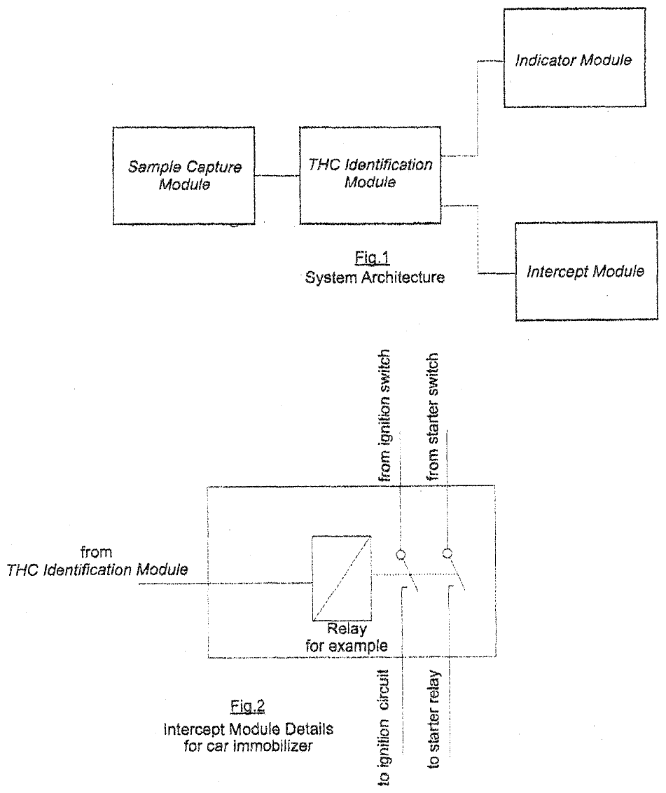

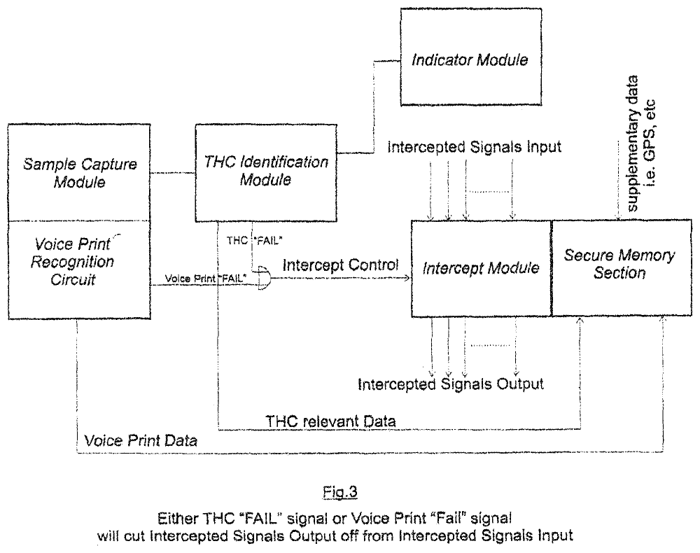

Indicator Modules or Indicator Devices may be lights, display assemblies(s), sirens, horns, speakers, or devices that shake or vibrate etc. Intercept Modules or Intercept Devices are inserted at a location where a conductor of electricity or light has been cut, and the cut can be bridged under the control of a THC Identification Module, to prevent the operating of equipment or access to locations, as shown in FIG. 2.

THC identification Modules may contain technology which is insensitive to common contaminants, and therefore may operate without some of the features currently in the Sample Capture Module. In principle, however, breath sample capture, THC Identification and one of or both of indicator and intercept technologies will be combined. The combination can, but does not have to, he in the form of all modules being co-located, although breath sample capture and sample evaluation via THC Identification will most likely he in close physical proximity. Transmission of the result derived by the THC identification module to Indicator and/or Intercept Modules can be over larger distances via electrical or optical signals.

Typical examples are on one hand a breathalyzer designed for road tests, which would combine sample capture, THC identification, and indicator in one handheld unit. In the case of a vehicle immobilizer the device would combine sample capture and THC identification in one handheld unit available to the driver of the car, probably combined with an indicator device to signal it being free to drive or not, and a remote mounted intercept module which receives a go/no-go signal, perhaps also quantitative THC information from the THC Identification Module for data logging, and, accordingly, allows or disallows energizing vehicle circuits necessary to operate the vehicle. One such signal is the electrical current sent from the ignition key to the ignition circuit in the case of gasoline engines. Another signal is from the starter key to the starter relay.

To support enforcement provisions would be made to log this information, together with date and time, in secure electronic memory that can be accessed by enforcement agencies.

It can generally be expected that some operators, who are under obligation to only operate such equipment that is outfitted with THC Identification and Intercept technology, will attempt to find ways to circumvent the blocking of vehicle operations, perhaps by taking the breath sample from another person, or by feeding the Sample Capture Module with fresh air in some clever way. One way to reduce the chances of that being successful would be to integrate a voice print recognition feature in the Sample Capture Module, the result of which could be stored in memory, as well as be combined, into the intercept Control signal, as may be seen in FIG. 3.

From the foregoing, it will be seen that this invention is one well adapted to attain the ends and objectives herein set forth, together with other advantages which are obvious, and which are inherent to the apparatus. As many possible embodiments, may be made of the invention without departing from the scope of the claims, it is to be understood that all matter herein set forth or shown in the accompanying drawings is to be interpreted as illustrative and not in a limiting sense. It will be appreciated by those skilled in the art that other variations of the preferred embodiment may also be practised without departing from the scope of the invention.

1. A controlled substance intercept system for detecting levels of one or more controlled substances in the breath of a subject and intercepting electric operation of a device, comprising:
a sample capture module;
a THC identification module; and
an intercept module; wherein the THC identification module sends an electric or optical signal to the intercept module if a level of controlled substance present in a sample captured in the sample capture module exceeds a set threshold.

2. The controlled substance intercept system of claim 1, further comprising an indicator module.

3. The controlled substance intercept system of claim 2, wherein the indicator module is selected front the group of indicators comprising: lights, display assemblies, sirens, horns, speakers, and vibrating devices.

4. The controlled substance intercept system of claim 1, wherein the sample capture module comprises means for removal of water from the breath, means for measuring the volume of the breath, and valve means for preventing withdrawal of the breath sample by the subject during inhalation.

5. The controlled substance detection system of claim 1, wherein the THC identification module comprises a controlled substance detection method selected from the group of controlled substance selection methods comprising: Asymmetric Waveform ion Mobility Spectrometry (FAIMS), Mass Spectrometry, Liquid Chromatography, Ion Resonance Chamber, Magnetic Resonance Chamber, Light Spectroscopy, Color detection, and Filtering.

6. The controlled substance detection system of claim 1, wherein the intercept module comprises means for interrupting the flow of electricity in a device.

7. The controlled substance intercept system of claim 1, wherein the intercept module includes a vehicle immobilizer.

8. The controlled substance intercept system of claim 1, wherein the intercept module includes an electronic door lock.

9. The controlled substance intercept system of claim 1, wherein the intercept module is remote from the sample capture module and THC identification module.

10. The controlled substance intercept system of claim 1, further comprising a voice recognition component for subject identification confirmation.

11. The controlled substance intercept system of claim 1, wherein the sample capture module includes a pressure regulator for regulating the pressure of the breath introduced into the system.

12. The controlled substance intercept system as recited in claim 11, in which the pressure regulator comprises an inflatable balloon to regulate the pressure of the breath introduced into the system.

13. A method of intercepting equipment operation in response to the presence of cannabis or other controlled substance by analyzing breath of a subject comprising the steps of:
removing contaminant material from the breath;
collecting a sample of at least one component of the breath of the subject, after the contaminant material has been removed therefrom;
analyzing the sample for the presence of one or more controlled substances;
sending a control signal to an immobilizer if the controlled substance is detected; and
immobilizing the equipment.

14. A method as recited in claim 13, further comprising the step of allowing the subject to inhale and exhale while introducing breath into the system.

15. A method as recited in claim 13, further comprising the step of regulating the pressure of the breath introduced into the system by the subject.

16. A method as recited in claim 13, further comprising the step of measuring a volumetric amount of breath introduced into the system by the subject.
